(19) United States
(12) Patent Application Publication
(10) Pub. No.: US 2010/0326441 A1
(43) Pub. Date: Dec. 30, 2010

(54) NASAL INTERFACE DEVICE

(76) Inventors: Shlomo ZUCKER, Michmoret (IL); Menachem Dvir, Herzelia (IL)

Correspondence Address: YORAM TSIVION, P.O. BOX 1307, PARDES HANNA, 37111, IL

(21) Appl. No.: 12/490,347

(22) Filed: Jun. 24, 2009

(51) Int. Cl. A61M 16/08

(52) U.S. Cl. 128/204.18; 128/207.18

(57) ABSTRACT

A nasal interface device connectable to a main air supply of a forced airway ventilation nasal system is disclosed. The nasal interface device includes at least a wye hose member having an inlet. The wye hose member is characterized by having a continuum forming portion, by having at least two hinge forming portions and by having two nostril associated portions so that the hinge forming portions of the wye hose member form a hinge of the nasal interface device, and upon compressing the continuum forming portion, the angle between the two nostril associated portions around the hinge is increased.

### FIELD OF THE INVENTION

The present invention relates in general to a nasal interface device. More particularly the present invention relates to a nasal interface device for nasal ventilation systems employed to deliver a breathing gas for human beings, through which breathing gas or air are directed to the nasal passages and breathed; the nasal interface device includes a hinge.

### BACKGROUND ART

U.S. Pat. No. 5,113,857 is believed to represent the state of the art.

### SUMMARY OF THE INVENTION

The nasal interface device of the present invention is to be used as a gas delivery terminal for any airway ventilation nasal system, employed to deliver a breathing gas into human beings, inter alia including forced airway ventilation nasal systems. Through the nasal interface device a breathing gas or air is directed into the nasal passages of a human being. The nasal interface device of the present invention is usable by forced breathing or forced airway ventilation nasal systems such as a continuous positive airway pressure (CPAP) systems.

The nasal interface device of the present invention is a structural element having one inlet for gases and two outlets for the incoming gas. The two outlets match the anatomical structure of the nose, to fit the two nostrils and employ a novel type of adaptive mechanism to accommodate for variations which may exist in the nasal structure. A central feature in this respect is a hinge which provides for an ergonomic affixing of the device onto the nose of a user.

### BRIEF DESCRIPTION OF THE DRAWINGS

The present invention will be understood and appreciated more fully from the following detailed description taken in conjunction with the appended drawings in which:

FIG. 1 is an isometric view of the nasal interface device of the present invention;

FIG. 1A is a cross-sectional isometric view of the wye hose member of the nasal interface device of the present invention;

FIG. 2 is yet another cross-sectional isometric view of the wye hose member of the nasal interface device of the present invention.

FIG. 2A is an enlarged view of adjustment lever and its environment;

FIG. 3 is an exploited isometric view of nasal interface device of the present invention;

FIG. 3A is an enlarged exploded view of the adjustment mechanism;

FIG. 4 is an isometric view of nasal interface device of the present invention;

FIGS. 5A-D showing isometric views of gasket, linear connector, right angle connector and U-shaped connector, according to some preferred embodiments of the present invention;

FIG. 6 showing an isometric view of the nasal interface device according to a preferred embodiment of the present invention;

FIGS. 7 to 8A respectively showing an isometric cross-sectional view, isometric exploded view and an enlarged view thereof of nasal interface device, according to a preferred embodiment of the present invention.

### DISCLOSURE OF THE INVENTION

Illustrative embodiments of the invention are described below. In the interest of clarity, not all features of an actual implementation are described in this specification. It will be appreciated that in the development of any such actual embodiment, numerous implementation-specific decisions must be made to achieve the developers' specific goals, such as compliance with system-related and business-related constraints, which vary from one implementation to another. Moreover, it will be appreciated that such a development effort might be complex and time-consuming, but would nevertheless be a routine undertaking for those of ordinary skill in the art having the benefit of this disclosure.

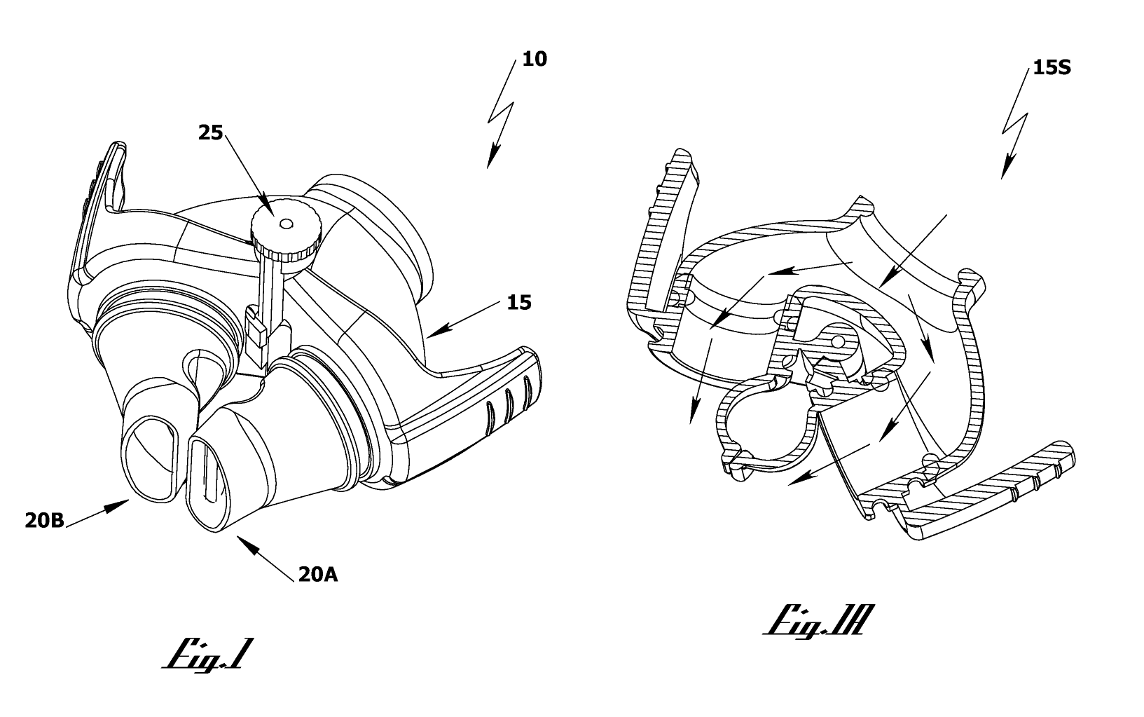

Reference is now made to FIG. 1 showing an isometric view of nasal interface device 10 according to a preferred embodiment of the present invention. Wye hose member 15 is furnished with nostrils engaging members (hereinafter NEMs) 20A and 20B and has adjustment mechanism 25 for adjusting the tolerance gap between the nasal septum engaging plates, as will be elaborated infra.

Reference is now made to FIG. 1A, showing a sectional isometric view of section 15S of the wye hose member of the nasal interface device of the present invention. The arrows on FIG. 1A indicate the bifurcating flow path of gases within the continuous portion of the wye hose member of the nasal interface device; from the one inlet thereof, for the incoming gas, and towards the two outlets thereof and further into the respective NEMs (not shown).

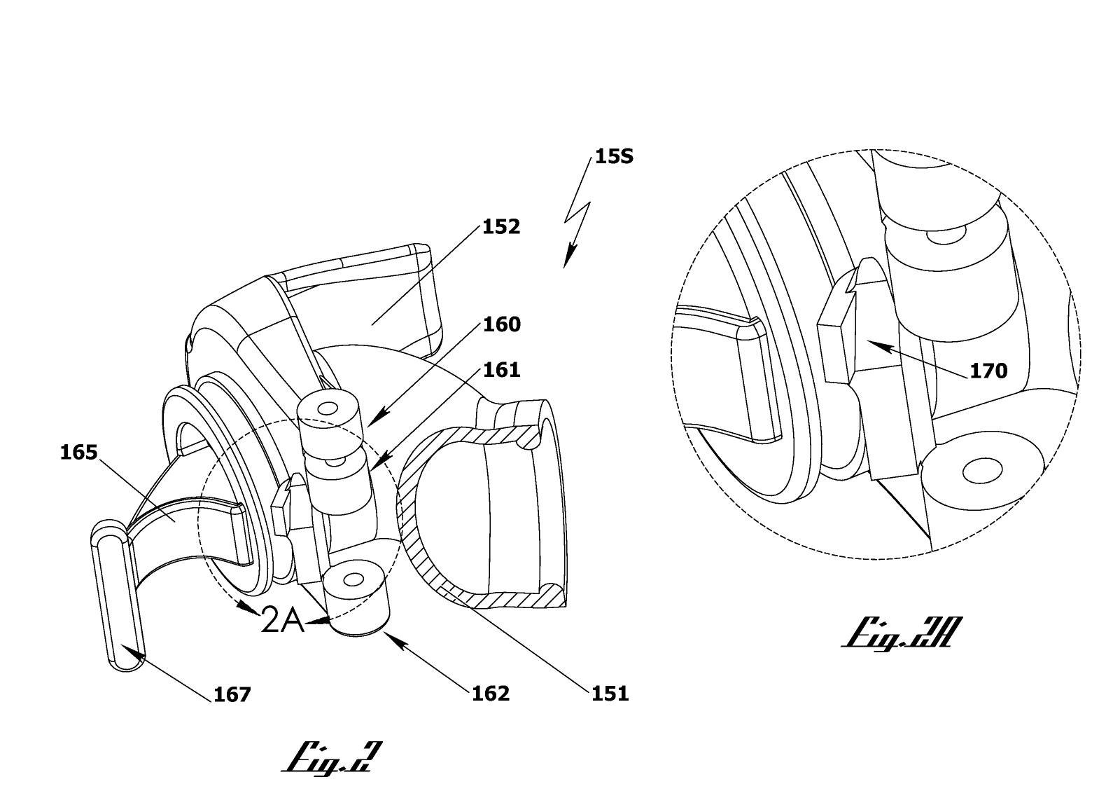

Reference is now made to FIG. 2, showing a sectional isometric view of section 15S of the wye hose member of the nasal interface device of the present invention. A row of tubular portions 160, 161 and 162 are disposed in tandem leaving spaces for matching interleaving tubular portions of the respective section (not shown), in such a way as the lumens of all tubular portions are alignable so that a pivot can be inserted therethrough, thereby forming a hinge. Section 15S further includes handle wing 152 and nostril associated portion 165 terminating with nasal septum engaging plate 167. Although the wye hose member is in essence monolithic, it is somewhat deformable as to allow adjustment of the angle between nostril associated portions thereof, as will be explained below.

Reference is now made to FIG. 2A showing an enlarged view of adjustment lever 170 and its environment. Lever 170 is a portion the wye hose member of the nasal interface device and it is used to adjust the tolerance gap between the nasal septum engaging plates, as will be explained infra.

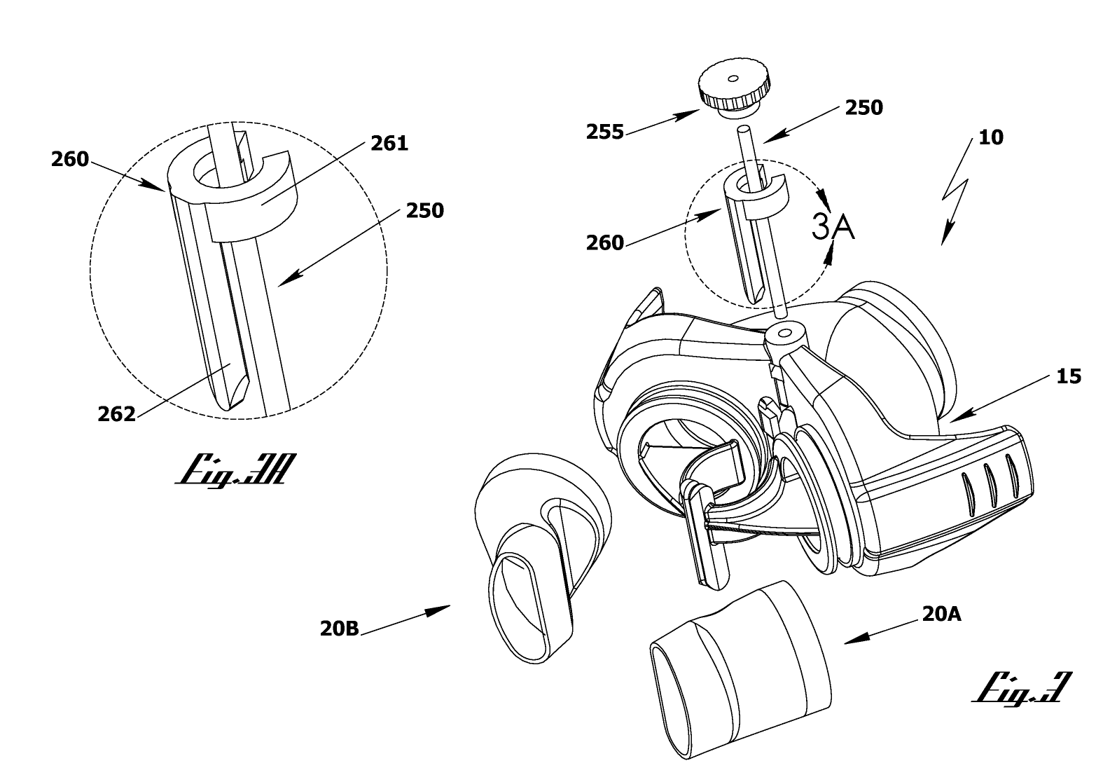

Reference is now made to FIG. 3 showing an exploited isometric view of nasal interface device 10. Wye hose member 15 is typically made of a firm resilient material capable of being slightly buckled or deformed. NEMs 20A and 20B are typically made of an elastic resilient material and can be mounted on the respective cylindrical extensions of wye hose member 15. NEMs 20A and 20B are to be inserted into the nostrils of a user. The elasticity/resiliency of NEMs 20A and 20B provides for an expansion thereof within the nostrils of a user, due to an inflation thereof prompted by a positive pressure within device 10; thereby the outer surfaces of NEMs 20A and 20B sealingly adjoin the inner surfaces of user's nostrils.

Reference is now made to FIG. 3A showing an enlarged exploded view of adjustment mechanism 25. Adjustment mechanism 25 includes hinge pivot 250 operationally connected with button cap 255 and wedging member 260. Wedging member 260 has cylindrical portion 261 to be coaxially mounted on top of the upper tubular portions of the wye hose member of the nasal interface device, and axial portion 162 used to exert a force onto adjustment lever of the wye hose member.

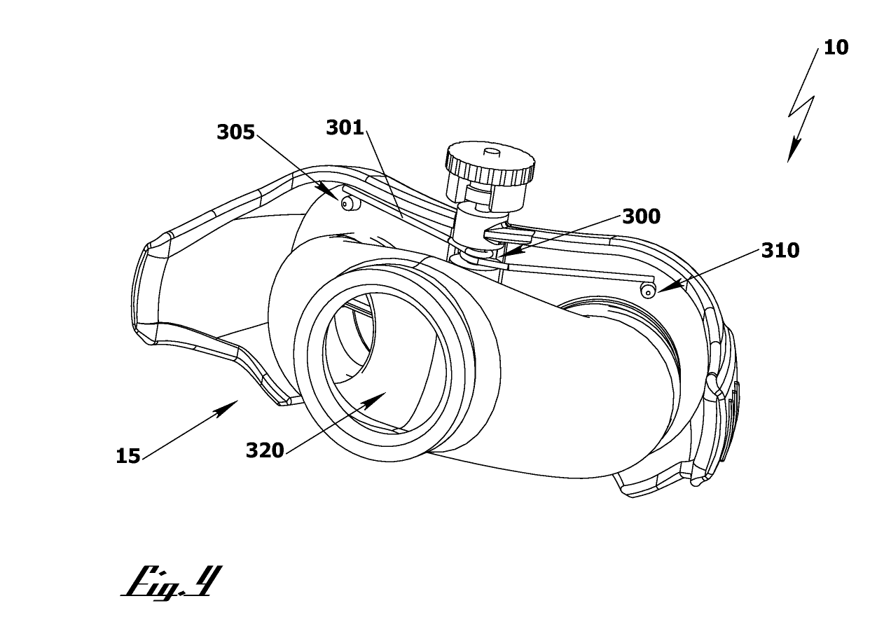

Reference is now made to FIG. 4 showing an isometric view of nasal interface device 10 according to a preferred embodiment of the present invention. Nasal interface device 10 has gap 300 in the hinge portion thereof; wherein biasing member 301 can be introduced. Biasing member 301, position of the terminal of which is secured pins 305 and 310, is used to exert a force onto wye hose member 15; thereby causing the nostril associated portions thereof to tighten around the hinge of device 10. Aperture 320 of wye hose member 15 is the inlet for accepting incoming gasses and for accomplishing that the end thereof can be furnished with various gaskets and/or connectors used to connect device 10 to any forced breathing or forced airway ventilation nasal system such as a continuous positive airway pressure (CPAP), or any type of breathing and/or ventilation system for that matter.

To explain how the nasal interface device 10 is used, reference is now made to FIGS. 1-4. In order to apply nasal interface device 10, the user pushes handle wings of wye hose member 15, such as handle wing 152; thereby expanding nostril associated portions, such as portion 165, of wye hose member 15 radially around the hinge of nasal interface device 10. Continuous bifurcating portion 151 of wye hose member 15 is compressed and sustains gentle deformation during the radial expansion of the nostril associated portions of wye hose member 15. As a result of the radial expansion of wye hose member 15 the clearance between nasal septum engaging plates, such as plate 167, of nostril associated portions of wye hose member 15, such as portion 165, is increased and the user can consequently introduce NEMs 20A and 20B into his/her nostrils. The user subsequently releases the handle wings of wye hose member 15 and the clearance between nostril associated portions is decreased; thereby the nasal septum engaging plates are fastened on his/her nasal septum. The user can adjust the tolerance gap between the nasal septum engaging plates by radially and/or axially translating wedging member 260 that exerts a force onto adjustment levers of wye hose member 15, such as adjustment lever 170; thereby causing increment/decrement of the aforementioned tolerance and providing for adjusting nasal interface device 10 to fit the thickness of the nasal septum of an individual user.

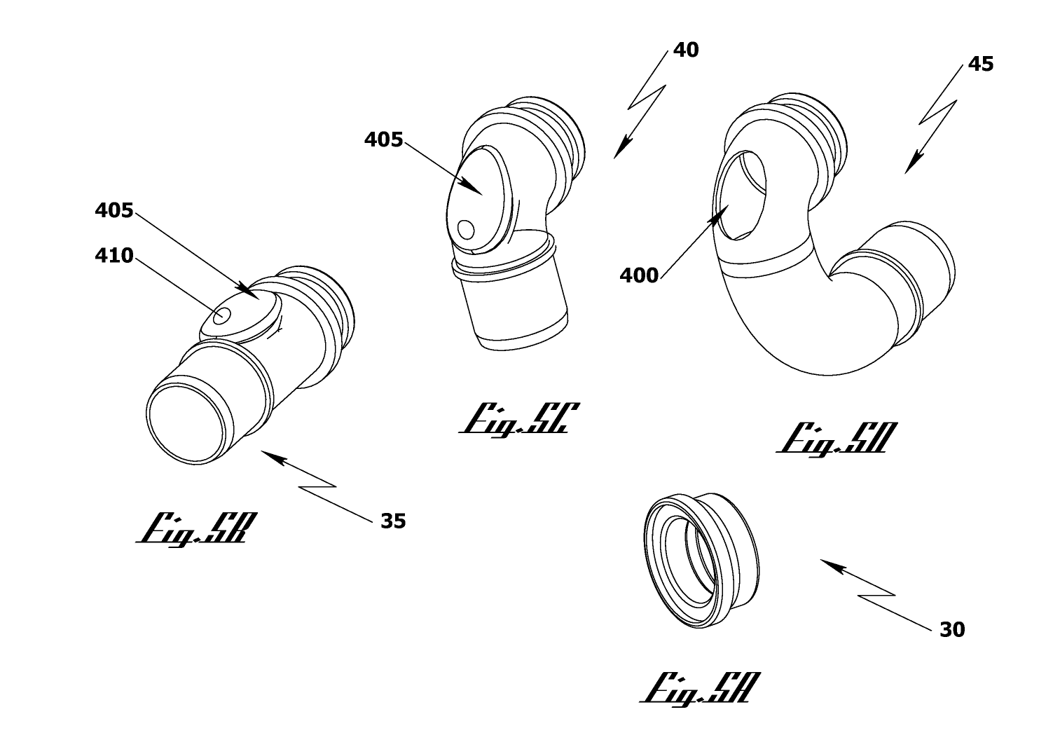

Reference is now made to FIGS. 5A-D showing isometric views of gasket 30, linear connector 35, right angle connector 40 and U-shaped connector 45, according to some preferred embodiments of the present invention.

Gasket 30 is mounted onto the inlet of the nasal interface device so that the rim thereof sealingly engages the exterior of the inlet of the device, whereas into the aperture in the centre of gasket 30 a connector, such as connectors 35, 40 or 45, or a tube are introduced.

Connectors 35, 40 and 45 are characterized by having elliptic aperture 400 covered by respectively matching lid 405. Elliptic aperture 400 and matching lid 405 can serve the purpose of a safety valve released at predetermined pressure threshold. Alternatively elliptic aperture 400 and matching lid 405 serve the purpose of an outlet for the air/gases exhaled by the user. Lid 405 has aperture 410.

### BEST MODE FOR CARRYING OUT THE INVENTION

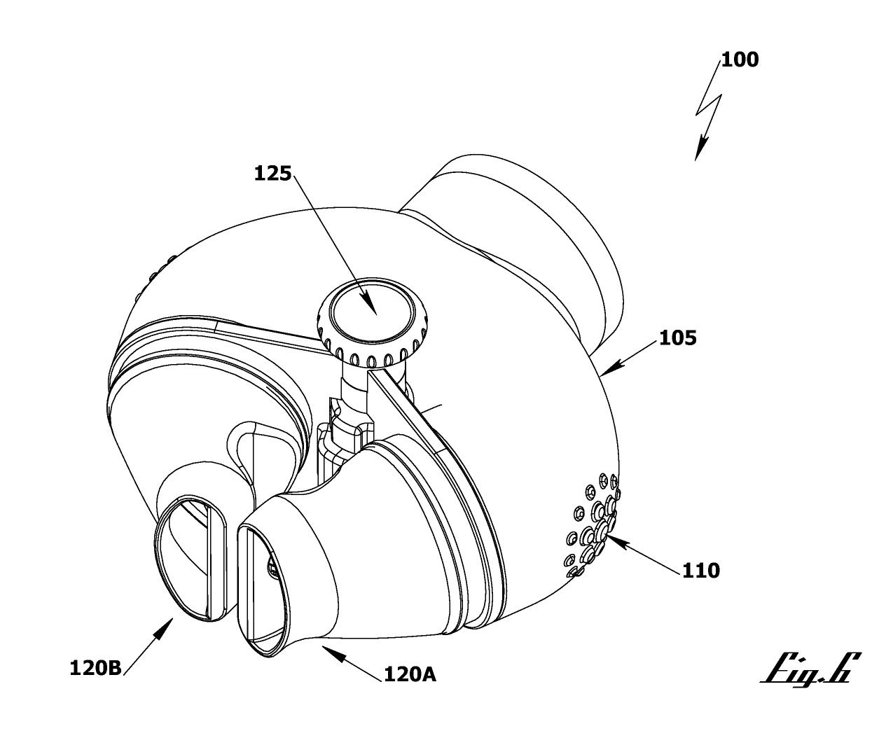

Reference is now made to FIG. 6 showing an isometric view of nasal interface device 100 according to a preferred embodiment of the present invention. Wye hose member 105 having textured suffice 110 is furnished with nostrils engaging members (hereinafter NEMs) 120A and 120B and has adjustment mechanism 125 for adjusting the tolerance gap between the nasal septum engaging plates, as will be elaborated infra.

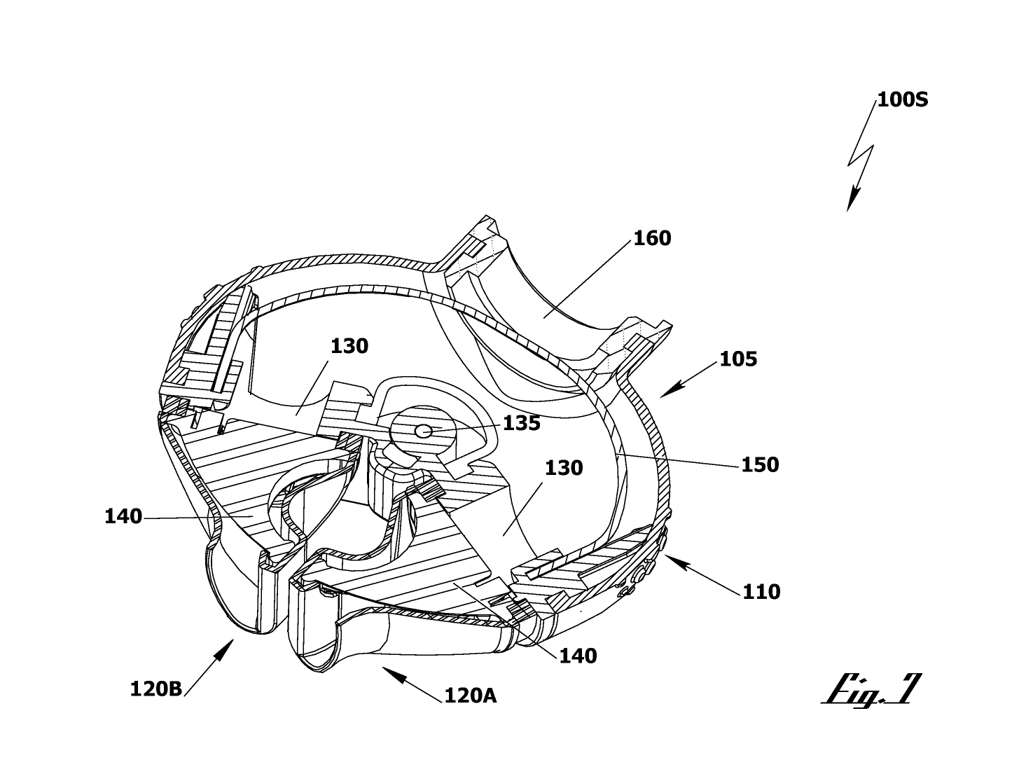

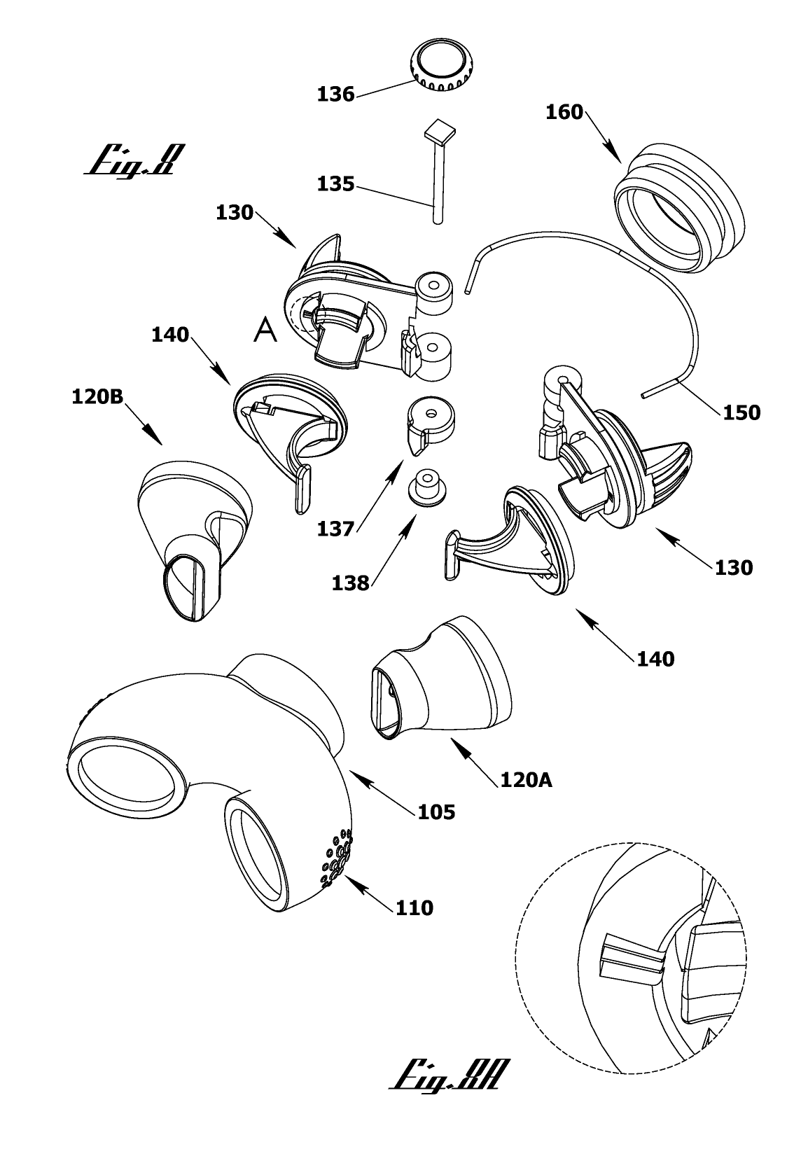

Reference is now made to FIGS. 7 to 8A, respectively showing a isometric cross-sectional view 100S, isometric exploded view and an enlarged view thereof of nasal interface device 100. Device 100 comprises core members 130 pivoted about pivot 135. Pivot 135 is furnished with knob 136. Pivot 135 is threaded into tubular portions of core members 130 which are interposed with wedging member 137, thereby forming a hinge. Pivot 135 is secured by tightening nut 138.

NEMs supporting members (henceforth NSMs) 140 are mounted onto core members 130 being controllably rotatable relatively thereto. The rotation of NSMs 140 is restricted by an interaction of V-shaped grooves within core members 130, shown in FIG. 8A, and respective detent (not shown, within NSMs 140. The rotation of NSMs 140 relatively to core members 130 provides for adjusting the tolerance angle between the nasal septum engaging plates of NSMs 140.

Although the wye hose member is in essence monolithic, it is somewhat deformable as to allow adjustment of the angle between NSMs 140. Wye hose member 105 is typically made of a firm resilient material capable of being slightly buckled or deformed. NEMs 120A and 120B are typically made of an elastic resilient material and can be mounted on the respective cylindrical extensions of NSMs 140. NEMs 120A and 120B comprise a pocket at the distal portion thereof adapted to confine the nasal septum engaging plates of NSMs 140; thereby provide for a more resilient conformation of NEMs 120A and 120B.

NEMs 120A and 120B are to be inserted into the nostrils of a user. The elasticity/resiliency of NEMs 120A and 120B provides for an expansion thereof within the nostrils of a user, due to an inflation thereof prompted by a positive pressure within device 100; thereby the outer surfaces of NEMs 120A and 120B sealingly adjoin the inner surfaces of user's nostrils.

Adjustment mechanism 125 includes wedging member 137. Wedging member 137 has cylindrical portion coaxially disposed in-between of the portions of core members 130 and axial portion used to exert a force onto adjustment lever of core members 130.

Nasal interface device 100 comprises biasing member 150. Biasing member 150 is used to exert a force onto core members 130; thereby causing NSMs 140 to tighten around the hinge of device 100. The inlet of wye hose member 105 for accepting incoming gasses is furnished with fitting 160 used to connect device 100 to any forced breathing or forced airway ventilation nasal system such as a continuous positive airway pressure (CPAP), or any type of breathing and/or ventilation system for that matter.

Device 100 is operated essentially as device 10, with the addition of the rotation of NSMs 140 relatively to core members 130 provides for adjusting the tolerance angle between the nasal septum engaging plates of NSMs 140.

It will be appreciated that the present invention is not limited by what has been particularly described and shown hereinabove and that numerous modifications, all of which fall within the scope of the present invention, exist. Thus for instance the nasal interface device may not include adjustment mechanism or the NEMs can be a continuum of wye hose member and not individual elements. Rather the scope of the invention is defined by the claims which follow:

1. A nasal interface device, connectable to a main air supply of a forced airway ventilation nasal system, said nasal interface device comprises at least a wye hose member having an inlet; said wye hose member is characterized by bifurcating the path of gases flowing therein, by having at least two tubular portions, and by having two nostril associated portions;
wherein said tubular portions of said wye hose member interconverge forming a hinge of said nasal interface device; and wherein upon compressing said wye hose member, the angle between said two nostril associated portions around said hinge is increased.

2. The nasal interface device as in claim 1, wherein said wye hose member is in essence a monolithic part, and wherein said tubular portions thereof are assembled into a hinge by introducing a pivot therethrough.

3. The nasal interface device as in claim 1, wherein said wye hose member comprises two core members and two NEMs supporting members.

4. The nasal interface device as in claim 3, wherein said two nostril associated portions are said two NEMs supporting members.

5. The nasal interface device as in claim 3, wherein said NEMs supporting members are mounted onto said core members controllably rotatable relatively thereto; thereby providing for adjusting the tolerance angle between the nasal septum engaging plates of NSMs.

6. The nasal interface device as in claim 1, wherein said wye hose member forming a bifurcating conduit from said inlet of said wye hose member towards said nostril associated portions thereof.

7. The nasal interface device as in claim 1, further comprising a constituent selected from the group consisting of:
a hinge pivot;
at least one nostrils engaging member (NEM);
a wedging member;
a button cap;
a biasing member.

8. The nasal interface device as in claim 7, wherein said NEM comprises a pocket at the distal portion thereof adapted to confine said nasal septum engaging plates.

9. A nasal interface device as in claim 1, wherein said wye hose member is further characterized by having at least one adjustment lever, and wherein the tolerance gap between said nostril associated portions can be adjusted by the means of said adjustment lever.

10. A nasal interface device as in claim 1, further comprising a constituent selected from the group consisting of:
a gasket adapted to said outlet;
a linear connector;
a right angle connector;
a U-shaped connector.
